(19) United States
(12) Patent Application Publication
(10) Pub. No.: US 2013/0248380 A1
(43) Pub. Date: Sep. 26, 2013

(54) Flexible Graphene Biosensor

(71) Applicant: UTAH STATE UNIVERSITY, North Logan, UT (US)

(72) Inventor: Yue Cui, Logan, UT (US)

(73) Assignee: Utah State University, North Logan, UT (US)

(21) Appl. No.: 13/850,877

(22) Filed: Mar. 26, 2013

Related U.S. Application Data
(60) Provisional application No. 61/615,737, filed on Mar. 26, 2012.

(51) Int. Cl. G01N 33/543

(52) U.S. Cl. CPC G01N 33/5438; Y10S 977/925; Y10S 977/734; Y10S 977/842; B82Y 40/00
USPC 205/777.5; 204/403.15; 204/403.14; 156/280; 977/925; 977/734; 977/842

(57) ABSTRACT

The present disclosure provides for a biosensor comprising a graphene electrode linked to a biosensing element by a linker, the biosensing element bonded to a flexible substrate. The graphene electrode has a first end and a second end, such that the first end may be a positive terminal and the second end a negative terminal. An electrical voltage may be applied to the positive and negative terminals to measure an electrical current response in proportion to a lactate concentration on the biosensing element. In embodiments, the biosensing element is an enzyme. By way of example, the biosensing element may be LOD.

### RELATED APPLICATIONS

This application claims benefit, under 35 U.S.C. §119(e), to U.S. provisional application No. 61/615,737, for “Flexible Graphene Sensor,” filed on Mar. 26, 2012, the entire contents of which are hereby incorporated by reference herein.

### TECHNICAL FIELD

The present disclosure relates to biosensors.

### BACKGROUND

Graphene is a single-atom-thick, sp² carbon-based material used in developing sensors and biosensors due to its remarkable electrical, optical, and mechanical properties. Graphene sensors and biosensors have been developed for highly sensitive detection of a variety of analytes, including nitric oxide, ammonia, hydrogen, glucose, and glutamate. Isolation of graphene has only recently been achieved, via epitaxial growth, chemical vapor deposition (CVD), chemical exfoliation, and mechanical exfoliation.

### SUMMARY

The present disclosure provides for a biosensor comprising a graphene electrode linked to a biosensing element by a linker, and bonded to a flexible substrate. The graphene electrode has a first end and a second end, such that the first end may be a positive terminal and the second end a negative terminal In embodiments, the biosensing element is an enzyme.

### BRIEF DESCRIPTION OF THE DRAWINGS

FIG. 1 illustrates a schematic of an enzyme-functionalized graphene electrode on a flexible substrate.

FIG. 2 shows an example of a current-time curve of a flexible biosensor of the present disclosure, to 1 μM, 2 μM, and 5 μM of lactate.

FIG. 3 shows a calibration curve for lactate with a flexible biosensor of the present disclosure (n=3).

FIG. 4 shows an example of the effect of bending angle on sensor response and graphene conductivity (lactate concentration: 10 μM), for a biosensor of the present disclosure.

FIG. 5 shows an example of the effect of bending number on sensor response and graphene conductivity (Lactate concentration: 10 μM. Bending angle: 180°), for a lactate biosensor of the present disclosure. The signal response (%) was calculated by normalizing the signal to the maximum signal obtained on the first measurement.

FIG. 6 shows SEM images of (a) graphene on Ni/SiO₂/Si wafer, (b) graphene on PET substrate, and (c) graphene on SiO₂/Si substrate.

FIG. 7 shows Raman spectra of graphene on Ni/SiO2/Si, with an excitation wavelength of 785 nm.

FIG. 8 shows Raman spectra of graphene on PET, with an excitation wavelength of 785 nm.

FIG. 9 shows the change in current response (%) to 1 μM lactate and graphene current with different bending angles.

FIG. 10 shows the change in current response (%) to 2 μM lactate and graphene current with different bending angles.

FIG. 11 shows the change in current response (%) to 5 μM lactate and graphene current with different bending angles.

FIG. 12 shows the effect of bending repetitions on current response to 5 μM lactate and graphene current for a bending angle of 180°.

### DETAILED DESCRIPTION

Rigid substrates of field effect transistors used in graphene biosensor construction limits the potential for wide range application of graphene biosensors. The applicant of the present disclosure has identified a need for a flexible graphene biosensor useful in healthcare, food testing, defense applications, environmental monitoring, or other fields where it is desirable to detect the presence or absence of an analyte. Due to the unique sensing properties of graphene, applicant also identified the use of graphene as a highly desirable means to develop wearable and flexible graphene biosensors that may be easily fabricated. Without limiting the embodiments of the present disclosure, applicant further determined that the controlled growth of graphene using CVD in a wafer scale on a metallic film, together with post-etching for graphene transfer, provide significant opportunities for the development of flexible graphene-based bioelectronics.

Lactate excreted in sweat and in blood is a biomarker for a variety of diagnostic purposes, such as heart failure, liver diseases, metabolic disorders, drug toxicity, and mortality in ventilated infants. Lactate in food can indicate microbial contamination, which may produce lactate fermentation. Due to the importance of detecting lactate, a variety of techniques have been investigated for its determination, including high-performance liquid chromatography, spectrophotometry, magnetic resonance spectroscopy, and amperometric biosensors based on Clark-oxygen electrodes or screen-printed electrodes. However, these methods are limited by time-consuming procedures, use of capital equipment, or the rigid nature of the devices, which are unsuitable, for example, for a variety of wearable, implantable, real-time, or on-site applications.

To address the problems identified by the applicant, the present disclosure provides for apparatuses and associated methods for making and using flexible graphene-based bioelectronics and biosensors. In embodiments, the biosensors may be bio-nanosensors.

In the following description, numerous specific details are provided for a thorough understanding of specific preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, structures, or characteristics may be combined in any suitable manner in a variety of alternative embodiments. Thus, the following more detailed description of the embodiments of the present invention, as illustrated in some aspects in the drawings, is not intended to limit the scope of the invention, but is merely representative of the various embodiments of the invention.

In this specification and the claims that follow, singular forms such as “a,” “an,” and “the” include plural forms unless the content clearly dictates otherwise. All ranges disclosed herein include, unless specifically indicated, all endpoints and intermediate values. In addition, “optional”, “optionally”, or “or” refer, for example, to instances in which subsequently described circumstance may or may not occur, and include instances in which the circumstance occurs and instances in which the circumstance does not occur. The terms “one or more” and “at least one” refer, for example, to instances in which one of the subsequently described circumstances occurs, and to instances in which more than one of the subsequently described circumstances occurs.

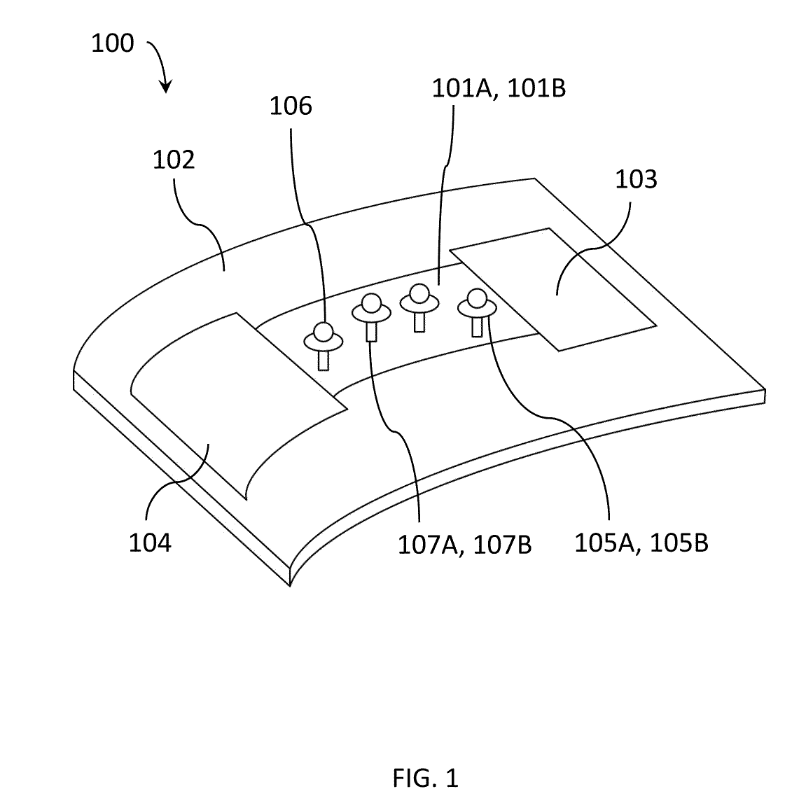

Referring to FIG. 1, the present disclosure provides for a flexible graphene biosensor 100 comprising a graphene electrode 101 with a first end and a second end, wherein one end of the graphene electrode 101 forms a first terminal 103 and the second end forms a second terminal 104, wherein, upon application of a voltage, the first terminal 103 and second terminal 104 provide for a positive terminal and a negative terminal The graphene electrode 101 is bonded to a flexible substrate 102. A biosensing element 105 capable of interacting with an analyte 106 is linked to the graphene electrode 101 by a linker 107.

Referring again to FIG. 1, a flexible substrate 102 may be any bendable substrate suitable for use with embodiments of the present disclosure. Preferably, the surface of the flexible substrate 102 is smooth enough to allow a graphene electrode 101 to bond to it without significantly reducing the conductivity of the graphene electrode 101. Flexible substrates 102 of the present disclosure may be chosen for unique combinations of electrical, thermal, chemical and mechanical properties that withstand extreme temperature, vibration or other demanding environments. By way of example, the flexible substrate 102 may be a plastic. In embodiments, the flexible substrate 102 may be a polyimide. In other embodiments, the flexible substrate 102 may be a polyester film. For example, without limiting the embodiments of the present disclosure, the flexible substrate 102 may be polyethylene terephthalate (PET). In alternative embodiments, the flexible substrate 102 may be a thermal release tape.

A graphene electrode 101 may have any number of graphene layers that provide the conductive properties required for embodiments of the present disclosure. The methods of constructing graphene electrodes 101 of the present disclosure may result in different numbers of graphene layers on different sections of a graphene electrode 101, and there is no requirement for uniformity of the graphene layering on graphene electrodes 101. Preferably, the number of graphene layers may be from one to six. More preferably, graphene electrodes 101 of the present disclosure may consist of four or fewer layers of graphene.

In embodiments, the biosensing element 105 may be an enzyme capable of binding to a linker 107, and also capable of interacting with an analyte 106, wherein the interaction between the analyte 106 and the biosensing element 105 provides a means to detect the presence or concentration of the analyte 106 in a sample. By way of example, the means of detection may involve a current response that arises as a result of an enzymatic reaction that occurs when the enzyme contacts the analyte. By way of further example, the biosensing element may be lactate oxidase (LOD).

In general, biosensors 100 of the present disclosure may be constructed by transferring graphene from a graphene source to a flexible substrate 102, and preparing terminals 103 and 104 of a graphene electrode 101A. Next, the graphene on the flexible substrate 102 is incubated with a linker 107A, at a suitable temperature and concentration, for a period of time sufficient to produce a linker modified graphene electrode 101B. Without limiting the invention, incubation may be for a period of about two hours, and may be carried out at room temperature. In embodiments, the linker modified graphene electrode 101B comprises a layer of linkers 107B bonded to the graphene electrode 101A. In embodiments, the linker 107 is a linker molecule. Without limiting the invention, the linker molecule may be 5 mM dimthylformamide. Next, the linker-modified graphene electrode 101B is incubated with biosensing elements 105A at a concentration and temperature sufficient to produce a layer of biosensing elements 105B covalently bound to the linker modified graphene electrode 101B. Following incubation, the linker modified graphene electrode 101B with a bound layer of biosensing element 105B may be rinsed. By way of example, the linker modified graphene electrode 101B may be incubated with 2 U μl⁻¹ of lactate oxidase in demineralized (“DI”) water overnight at 4° C., followed by rinsing with DI water and phosphate buffered saline solution (PBS) (0.1 M, pH 7.5).

Generally, a biosensor 100 of the present disclosure may be used to determine the concentration of an analyte 106 in a sample by measuring the current response generated by the interaction of biosensing elements 105 with an analyte 106. By way of example, the interaction of the biosensing elements 105 and the analyte 106 may produce a product that generates an electrical current response in the presence of an applied voltage. In embodiments, methods of the present disclosure for sensing the presence or determining the concentration of an analyte may be carried out by contacting an enzyme-functionalized, graphene electrode 101B of a biosensor 100 with a sample, measuring an electric current response in the presence of an applied voltage, and optionally correlating the electric current response to the level of an analyte 106 in the sample. Without limiting the embodiments of the present disclosure, the sample may be blood, sweat, tears, urine, culture medium, or any other suitable biological sample, and may be obtained from an animal, human, or microbial culture.

By way of example, measurements may be conducted using Autolab PGSTAT101 and carried out while the measuring device is biased at 300 mV. The measuring device may be used in combination with tNOVA software connected with a computer via USB interface for making the electrochemical measurements. Measurements may be carried out at any suitable temperature. Preferably, measurements may be performed at room temperature ˜19° C.). To carry out measurements, a sample containing an analyte 106 is applied to a graphene electrode 101 of a biosensor 100 of the present disclosure. An electrical current response is measured and is optionally correlated to the concentration of an analyte 106 in the sample.

The following examples are illustrative of specific methods to make and use biosensors 100 of the present disclosure, and are not necessarily intended to limit the embodiments of the present disclosure.

### EXAMPLES

### Example 1

### Flexible Lactate Biosensor

Generally, lactate biosensors of the present disclosure may be constructed by transferring a graphene electrode 101 from a rigid substrate to a flexible substrate 102, patterning with source 103 and drain electrodes 104, and immobilizing a specific enzyme for lactate on graphene. Due to the ultrathin layer of graphene, the biosensor 100 may detect lactate sensitively and rapidly. The flexibility of the substrate further allows for detecting lactate under different mechanical conditions.

Referring to FIG. 1, a flexible graphene biosensor 100 of the present disclosure was constructed by transferring graphene from a CVD chip to a flexible substrate 102 made of flexible polyester film (“PET”). The graphene on PET was then patterned with source and drain electrodes (silver paste, or Au). Lactate oxidase (LOD) was immobilized on the graphene electrode 101A by 1-pyrenebutanoic acid succinimidyl ester, with one end strongly attaching to graphene through π-π interactions with the pyrene group and the other end covalently bonding to the amino group of LOD with an amide bond. The enzyme and LOD catalyze lactate and oxygen to produce pyruvate and hydrogen peroxide (H₂O₂), according to the chemical reaction:

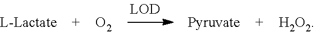

The oxidation of H₂O₂ on a graphene electrode 101 generates an electrical current response proportionate to the concentration of lactate. The measured electrical current response can be used to determine the concentration of lactate.

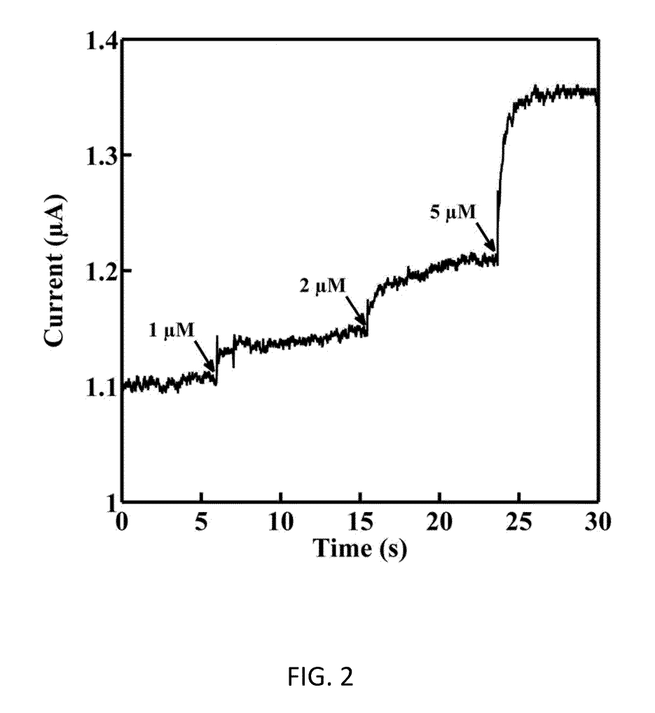

Referring to FIG. 2, an increase in current was observed when lactate was added to an enzyme functionalized graphene electrode 101B. The sensor response to 1 μM of lactate was 39±2.3 nA was significantly higher than the noise level 4±2.0 nA. The sensor response further increased when a higher concentration of lactate was used. The sensor response was proportional to the lactate concentration. The signal response of this biosensor 100 was rapid, and the steady-state background current increased after the addition of lactate and reached a new stationary state in about two seconds, which means that the total measurement using the biosensor 100 took only a few seconds.

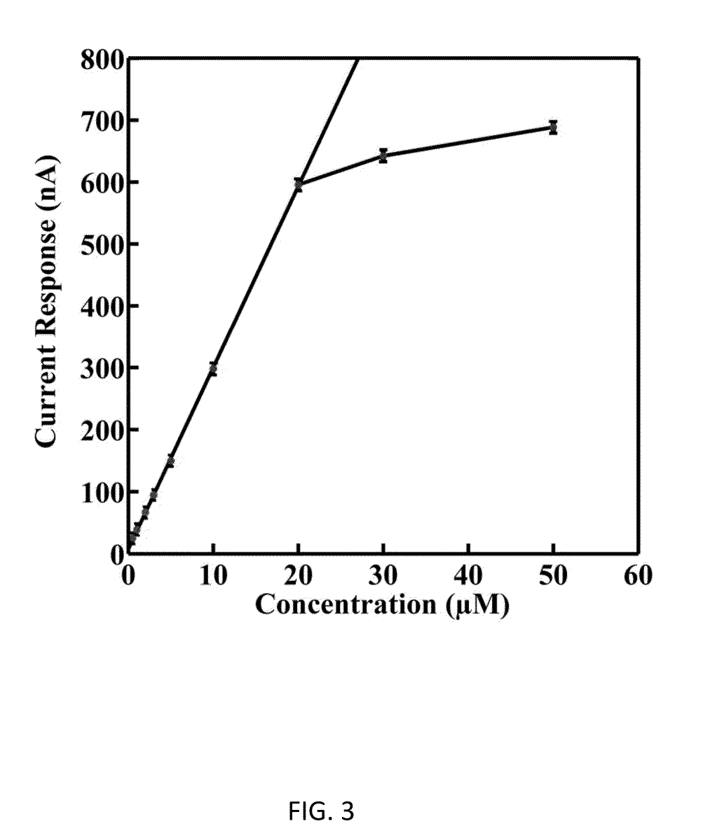

FIG. 3 illustrates a calibration curve for lactate with the flexible graphene biosensor 100. A linear relationship was obtained between the electrical current response and the concentration of lactate with a detection limit of 0.08 μM, a saturation concentration of 20 μM, and a slope of 29.869.0 nA μM⁻¹ (R2=0.999, n=3). The electrical current response was found to be stable and reproducible.

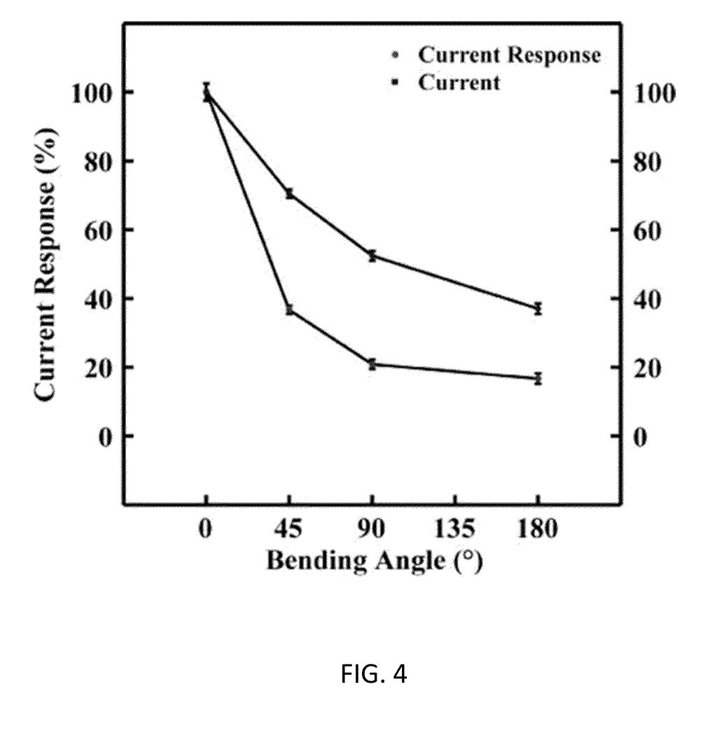

Referring now to FIG. 4, the effect of mechanical bending on the sensing performance of the enzyme-functionalized graphene on a flexible substrate 102 was examined Flexible graphene biosensors 100 of the present disclosure were bent to varying radii of curvature to induce tensile stresses, and the resulting changes in electrical properties and overall sensing performance were evaluated. The bending was applied to graphene electrodes 101B of newly prepared biosensors 100.

Still referring to FIG. 4, the effect of the bending angle on sensor response and graphene conductivity is shown. Generally, graphene conductivity decreased as bending curvatures increased. Graphene exhibited the highest sensor response and conductivity when unbent. The sensor response to 10 μM lactate decreased by 64% and the graphene conductivity decreased by 30%, with a 45° bending angle. The percentage of the decrease in sensor response was significantly higher than that in graphene conductivity. Sensor response and graphene conductivity decreased with higher bending angles. For example, an 84% decrease in the current response and a 63% decrease in graphene conductivity were observed with a 180° bending angle. These decreases may have resulted from changes in the surface morphology of the thin graphene layer. Bending causes reduced electron transport across the thin layer and thus reduced electrode conductivity and sensor sensitivity. During the mechanical bending of the flexible biosensor 100, the layer of enzymes 105 functionalized on the graphene electrode 101B is also modified, and changes in layer of enzymes 105 on graphene also contribute to reduced sensor sensitivity. Thus, the combined effect disruption to the layer of enzymes 105 and graphene layer damage may result in the reduced sensor response.

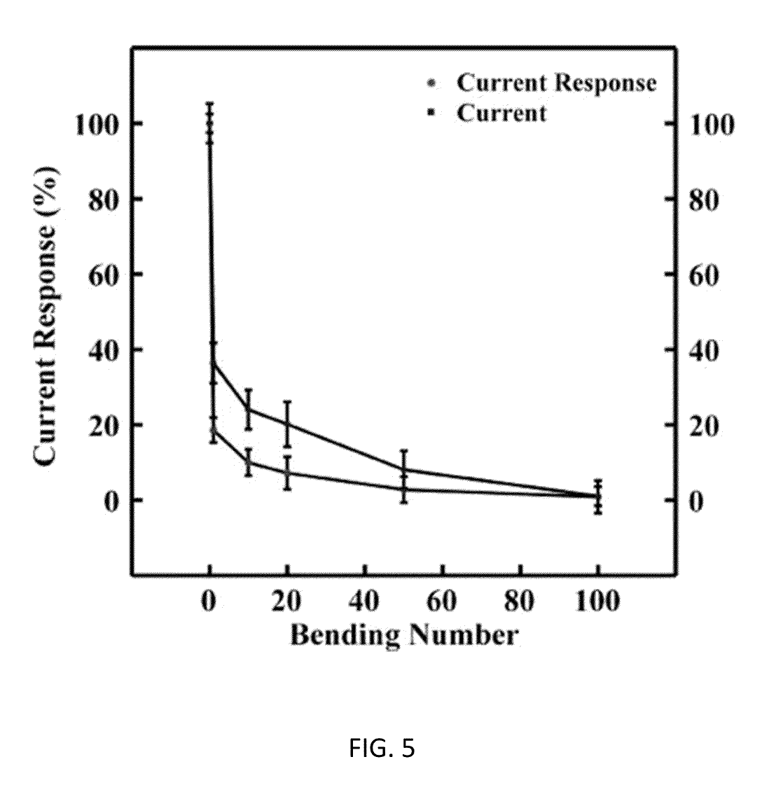

FIG. 5 illustrates the effect of repeated bending on the behavior of a flexible graphene biosensor 100 of the present disclosure, including the effect of bending repetitions upon the sensor response and graphene conductivity with extreme inward 180° bending, for the detection of 10 μM of lactate. Unbent graphene exhibited the highest sensor response and graphene conductivity, which further decreased with increasing bending repetitions. An 81% decrease of sensor response and a 64% decrease of graphene conductivity were observed following a single 180° bending. A 90% decrease of current response and a 76% decrease of graphene conductivity were observed with 10 repetitions. Similar changes for the lactate sensor signal and graphene conductivity were observed following more bending repetitions. Bending repetitions may affect the graphene layer significantly, and the decrease in conductivity may be attributed to damage to the graphene layer during bending. Reduced conductivity likely accounts for the majority of the decrease in sensor sensitivity shown in FIG. 5.

### Example 2

### Apparatus and Chemicals Used for a Lactate Biosensor

A potentiostat, Autolab PGSTAT101 (Metrohm USA, Riverview, Fla.) and a computer installed with Autolab NOVA software were used to measure the electrical response of the grapheme biosensor 100 under various conditions. The optical microscope was purchased from Microscopes, Inc. (Northbrook, Ill.). CVD graphene (CVD graphene on Ni film on SiO₂/Si) was purchased from Graphene Supermarket (Calverton, N.Y.). PELCO Conductive Silver 187 used as the terminals for the graphene electrode was purchased from Ted Pella. Inc. (Redding, Calif.). Epoxy was purchased from Epoxies, Etc. (Cranston, R.I.). Lactate oxidase was purchased from Toyobo Co., Ltd. (Osaka, Japan). L-(+)-Lactic acid was purchased from Sigma-Aldrich, Co. (St. Louis, Mo.). Thermal release tape (Revalpha thermal release tape, No. 319Y-4MS) was purchased from Nitto Denko America, Inc. (Fremont, Calif.). Polyester (PET) film (Melinex film ST507/200) was from Dupont Teijin films (Chester, Va.). Kapton tape was purchased from SRA Soldering Products (Foxboro, Mass.). Kapton (polyimide) films, Type VN (125 μm) and Type HN (50 μm), were purchased from American Durafilm (Holliston, Mass.). 1-Pyrenebutanoic acid succinimidyl ester was purchased from Anaspec, Inc. (Fremont, Calif.). N,N-Dimethylformamide 99% (DMF) was purchased from Acros Organics (Pittsburgh, Pa.). Ferric (III) chloride (FeCl₃), potassium phosphate monobasic, and potassium phosphate dibasic were from Fisher Scientific (Pittsburgh, Pa.). All the solutions were prepared in ultrapure water obtained from Barnstead NANOpure® DIamond™ Water Systems (Thermo Scientific, Asheville, N.C.).

### Example 3

### Transfer of Graphene to Prepare a Lactate Biosensor

To transfer graphene to a flexible substrate 102, a thermal release tape was attached to a CVD graphene chip (graphene on Ni/SiO₂/Si). The tape adhering to the substrate was then soaked in water with a gentle ultra-sonication. After a few minutes, the tape/graphene/Ni layers on the chip were peeled off from the SiO₂/Si substrate as water intervened between the Ni and SiO₂. The separated tape/graphene/Ni layers were then etched in FeCl₃ solution to remove the Ni layers, and the remaining graphene on thermal release tape was washed with ultrapure water and dried. This graphene was then transferred to the flexible substrate 102 by bringing the tape with graphene into contact with a flexible substrate and placing it on a hot plate at a temperature of 130° C., which is slightly hotter than the release temperature of 120° C. for the thermal release tape. Two silver-paste based terminals were used to contact the graphene electrode and were coated with epoxy for insulation in order to minimize possible interferences during sensing measurements.

### Example 4

### Enzyme Functionalization for a Lactate Biosensor

Graphene films were transferred to PET substrate. After preparing the positive terminal on a first terminal 103 and a negative terminal on the second terminal 104 on opposite sides of the graphene electrode 101A, the graphene film was incubated with a 5 mM linker molecule 107A (1-pyrenebutanoic acid succinimidyl ester) in dimethylformamide (DMF) for 2 hours at room temperature followed by washing with DMF and ultrapure water. The linker-modified graphene 107B was then incubated with 2 U μl-1 of lactate oxidase at 4° C. overnight, then rinsed with ultrapure water and phosphate buffered saline solution (PBS) (0.1 M, pH 7.5).

### Example 5

### Sensing Measurements with a Lactate Biosensor

Measurements were carried out with the measuring device biased at 300 mV. All measurements were performed at room temperature (˜19° C.). To understand the electrical behavior of a flexible biosensor when subjected to mechanical bending, the current responses of the biosensor were measured upon bending inward to angles of 0°, 45°, 90° and 180°. To facilitate electrical measurements during bending, the enzyme functionalized graphene electrode 101B on PET was placed horizontally between two stands, with external copper wire touching the terminals 103 and 104 on both sides of the graphene 101A. The graphene electrode 100 was bent at different angles by moving the two stands closer together or farther apart. Lactic acid droplets in increasing order of concentration were pipetted onto the graphene electrode strip 101B covering completely the cross section of the graphene area 101B, without contacting the silver-paste electrodes 103 and 104. Using this method, applicants were able to measure the change in current response resulting from different lactate concentrations at various bending angles.

### Example 6

### Substrates

Graphene films were successfully transferred onto different plastic substrates including Kapton films, Kapton tape and thermal release tape. The transfer process for these substrates was similar to that used for PET.

### Example 7

### Graphene

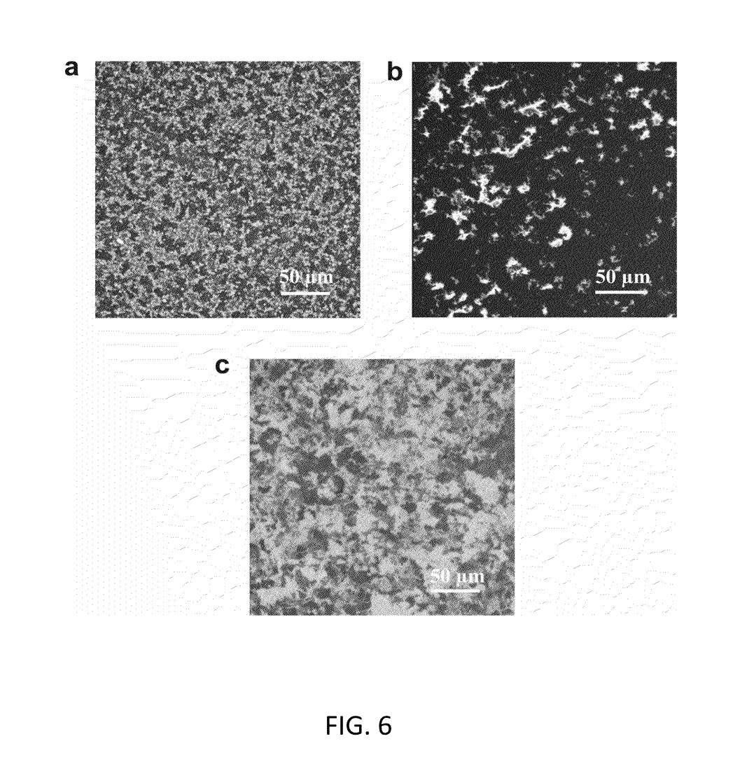

Referring now to FIG. 6a-6c, CVD graphene was grown on Ni/SiO₂/Si wafer, and graphene was transferred from Ni/SiO₂/Si to PET or SiO₂/Si. In FIG. 6a, the SEM image of graphene on Ni shows a few non-uniform layers of graphene. As shown in FIG. 6b, graphene was then transferred from a Ni/SiO₂/Si substrate to a PET substrate, due to the non-conductivity of PET, charging occurred in the SEM, making it difficult to produce a good SEM image. Therefore, transferring graphene from a Ni/SiO₂/Si substrate to a conductive substrate (SiO₂/Si) was further investigated in order to observe the transferred graphene more clearly. As shown in FIG. 6c, the transferred graphene on SiO₂/Si shows a similar morphology as the graphene grown on Ni/SiO₂/Si.

### Example 8

### Raman Spectra

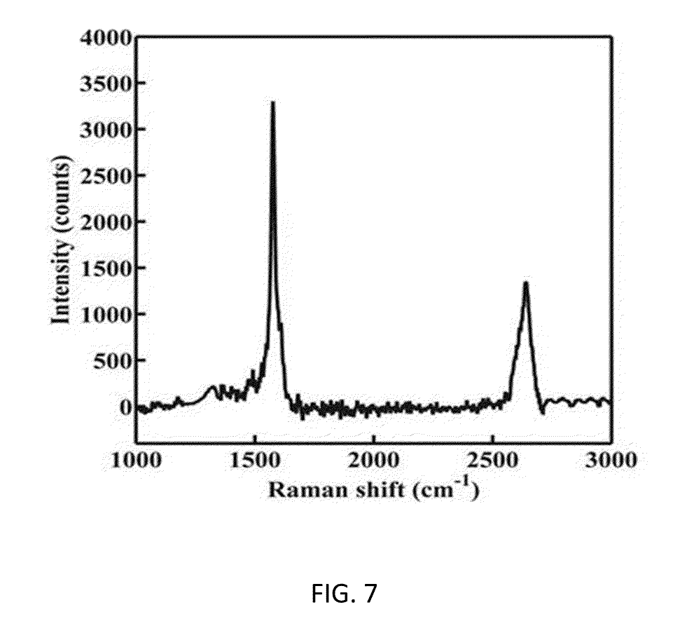

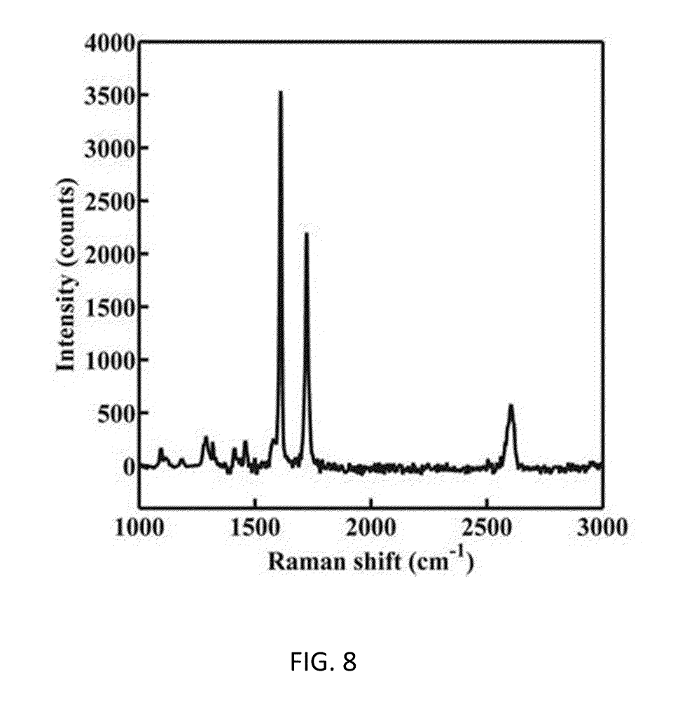

Referring now to FIGS. 7 and 8, Raman spectra were investigated for graphene on Ni/Si0₂/Si and graphene on PET. The measurements were performed at room temperature with a Renishaw spectrometer at 785 nm and a 50× objective. For both graphene on Ni/SiO₂/Si and graphene on PET, the characteristic G peak and 2D peak were observed at 1600 cm⁻¹ and ˜2600 cm⁻¹ individually. PET is a complex substrate for Raman analysis with a significant amount of background disturbance, and subtraction of background signals was performed in order to observe the peaks for graphene on PET with Raman analysis. Some background noise of PET cannot be fully removed by subtraction which overlaid the Raman peaks for graphene, noise peaks can be seen at ˜1300 cm⁻¹ and ˜1700 cm⁻¹, and the 2D Raman peak at ˜2600 cm⁻¹ obtained from graphene on PET was smaller than that from graphene on Ni/SiO₂/Si.

### Example 9

### Wearable Biosensors

In embodiments, the present disclosure provides for a biosensor useful as a wearable sensor. Flexible graphene biosensors of the present disclosure may be highly sensitive. Although sensitivity reduces significantly with increased bending angles and the number of times the sensor is bent, the sensor is still able to detect low concentrations of lactate sensitively and rapidly under different mechanical conditions. The sensor is suitable for use in a variety of wearable applications, including, but not limited to, monitoring lactate on skin.

### Example 10

### Bending Angles

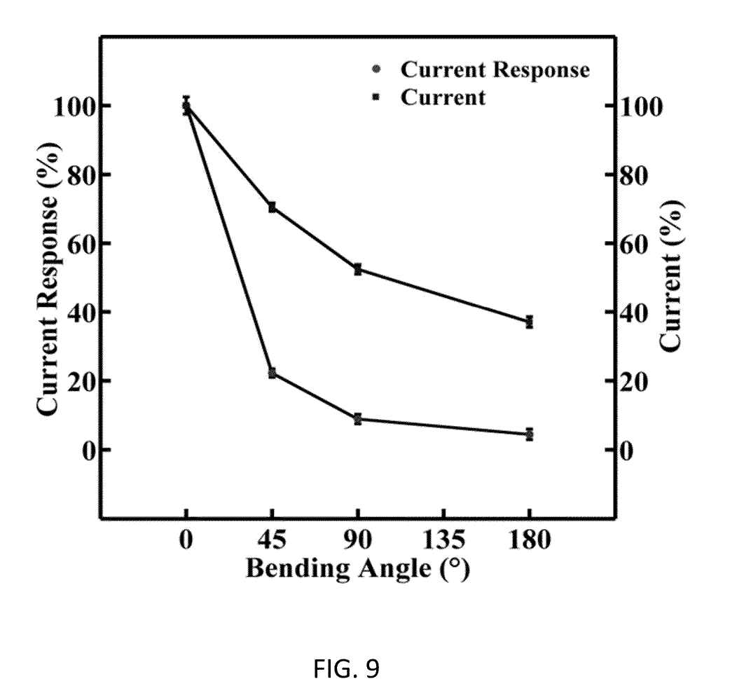

Referring now to FIG. 9, the effect of bending angle on a wearable graphene biosensor of the present disclosure was examined The effect of bending angle was investigated as previously discussed. FIG. 9 shows the change in current response (%) to 1 μM lactate and graphene current with different bending angles.

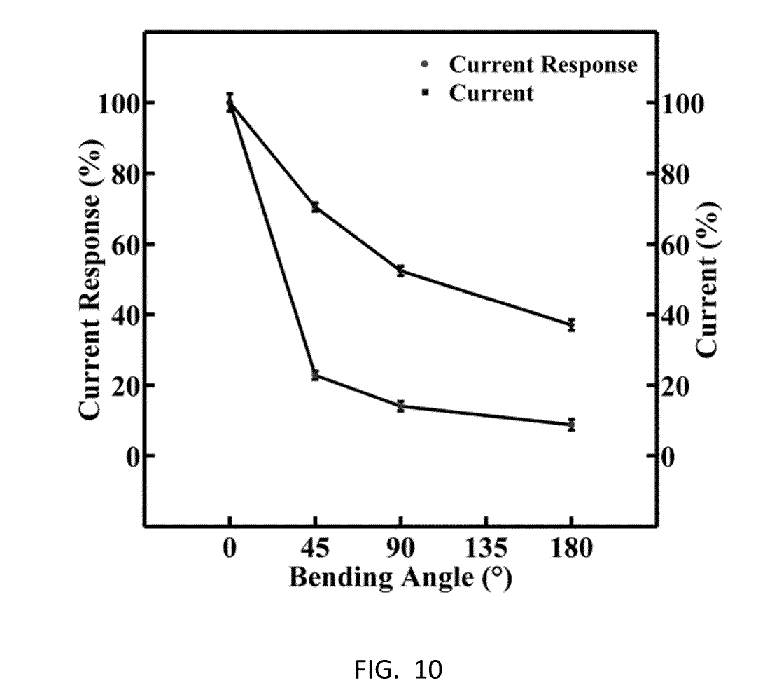

Referring now to FIG. 10, the effect of bending angle on a flexible graphene biosensor of the present disclosure was examined Shown is the change in current response (%) to 2 μM lactate and graphene current with different bending angles.

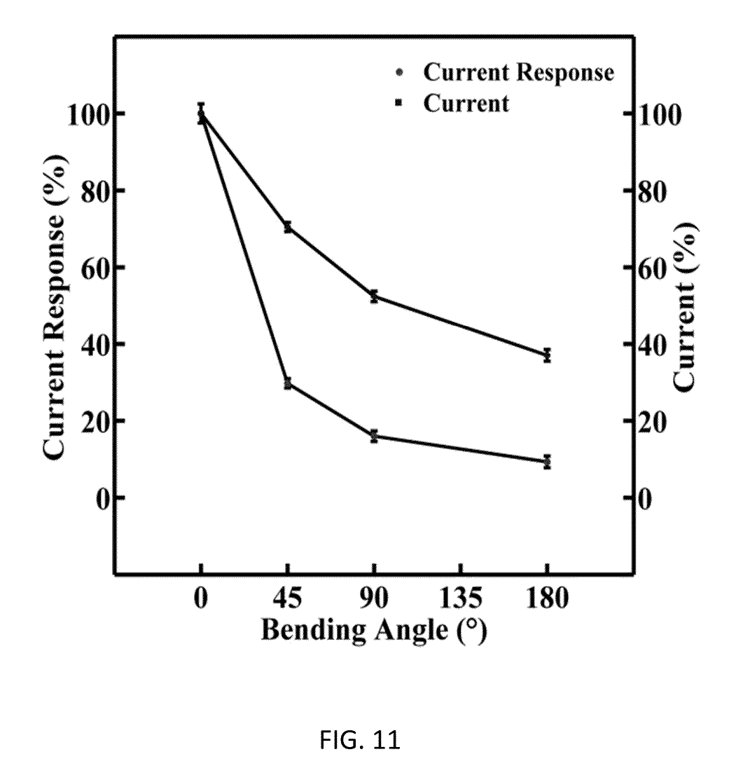

Referring now to FIG. 11, the effect of bending angle on a wearable graphene biosensor of the present disclosure was examined Shown is the change in current response (%) to 5 μM lactate and graphene current with different bending angles.

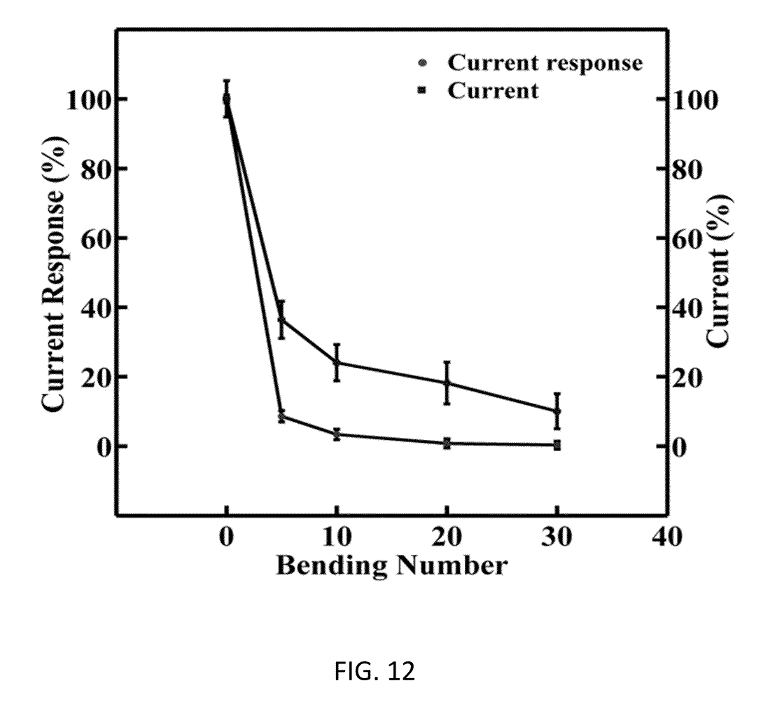

Referring now to FIG. 12, the effect of bending repetitions on current response for a flexible biosensor of the present disclosure was examined. Shown is the current response to 5 μM lactate and graphene current for a bending angle of 180°.

It will be appreciated that several of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

What is claimed is:

1. A biosensor, comprising:
a flexible substrate;
a graphene electrode bonded to the flexible substrate, the graphene electrode having a first end and a second end;
a first electrical terminal formed on the first end and a second electrical terminal formed on the second end; and
a biosensing element linked with a linker molecule to the graphene electrode between the first and second electrical terminals.

2. A biosensor of claim 1, wherein the flexible substrate is a plastic.

3. A biosensor of claim 1, wherein the flexible substrate is a polyester film.

4. A biosensor of claim 1, wherein the flexible substrate is a polyimide.

5. A biosensor of claim 1, wherein the flexible substrate is a polyester.

6. A biosensor of claim 1, wherein the flexible substrate is PET.

7. A biosensor of claim 1, wherein the flexible substrate is thermal release tape.

8. A biosensor of claim 1, wherein the biosensing element is a biological molecule.

9. A biosensor of claim 8, wherein the biological molecule is an enzyme.

10. A biosensor of claim 1, wherein the graphene electrode comprises from one to six layers of graphene.

11. A biosensor of claim 10, wherein the layers of graphene are non-uniformly distributed over the surface of the graphene electrode.

12. A biosensor of claim 1, wherein the biosensing element is an enzyme.

13. A biosensor of claim 12, wherein the enzyme is LOD.

14. A biosensor of claim 1, wherein the linker molecule is 1-pyrenebutanoic acid succinimidyl ester.

15. A method of manufacturing a biosensor, comprising:
transferring graphene from a graphene source to a flexible substrate to provide for a graphene electrode;
preparing a first terminal on a first end of the graphene electrode and a second terminal on a second end of the graphene electrode;
incubating the graphene on the flexible substrate with a linker to produce a linker modified graphene with a layer of linkers; and
incubating the linker modified graphene with a biosensing element to provide a functionalized graphene electrode.

16. The method of claim 15, wherein the flexible substrate is a plastic.

17. The method of claim 15, wherein the flexible substrate is a polyester.

18. The method of claim 15, wherein the flexible substrate is a polyimide.

19. The method of claim 15, wherein the flexible substrate is PET.

20. The method of claim 15, wherein the flexible substrate is thermal release tape.

21. The method of claim 15, wherein the biosensing element is an enzyme.

22. The method of claim 21, wherein the enzyme is LOD.

23. A method of sensing an analyte, comprising:
contacting a sample to an enzyme functionalized graphene electrode on a flexible substrate;
applying a known voltage across the enzyme functionalized grapheme electrode;
measuring a current response; and
correlating the current response to the level of a analyte in the sample.
